(19) United States
(12) Patent Application Publication
(10) Pub. No.: US 2024/0261496 A1
(43) Pub. Date: Aug. 8, 2024

(54) LIGHT PROTECTIVE INFUSION SET DEVICES

(71) Applicant: CAREFUSION 303, INC., San Diego, CA (US)

(72) Inventor: Lisa M. DAVIS, San Antonio, TX (US)

(21) Appl. No.: 18/271,425

(22) PCT Filed: Jan. 27, 2022
(86) PCT No.: PCT/US2022/014118

§ 371 Date: Jul. 7, 2023

Related U.S. Application Data
(60) Provisional application No. 63/152,176, filed on Feb. 22, 2021.

(51) Int. Cl. A61M 5/14; A61M 39/08

(52) U.S. Cl. CPC A61M 5/1415; A61M 39/08; A61M 2205/0238

(57) ABSTRACT

A light protective infusion set component comprising an infusion set component is provided. A portion of the infusion set component is translucent, providing visibility within the infusion set component; and a layer of flexible light protective material having a filter for a predetermined range of light wavelengths, wherein the layer of flexible light protective material is disposed around an exterior surface of the translucent portion of the infusion set component. Light protective infusion set layers and methods of forming a light protective infusion set component are also provided.

### CROSS-REFERENCE TO RELATED APPLICATION

The present application claims priority to U.S. Provisional Application No. 63/152,176, filed on Feb. 22, 2021, and entitled “LIGHT PROTECTIVE INFUSION SET DEVICES,” the entirety of which is incorporated by reference herein.

### TECHNICAL FIELD

The present disclosure generally relates to infusion set devices, in particular light protective infusion set devices.

### BACKGROUND

Typical infusion or intravenous (IV) sets are constructed by joining multiple translucent polymeric tubing segments to multiple polymeric components, many of which are also translucent. These IV sets are then used with infusion pumps or gravity systems to provide fluids to a user, such as a patient. However, many drugs, medical solutions and biomedical fluids are sensitive to light, where exposure to certain wavelengths of light (e.g., ultraviolet (UV) light) causes the drugs, medical solutions and biomedical fluids to degrade or transform. Such degradation or transformation may reduce the efficacy of the treatment or even introduce harmful substances into the patient.

In order to deal with this issue, some typical solutions are to provide opaque tubing or IV set components, but this solution prevents the fluid path or levels for these components from being visible. Other solutions have been to wrap IV sets, fluid sources and pump systems with aluminum foil, which again prevents visibility of the fluid path or level within the wrapped component, and further introduces the issue of making all of these components and systems electrically conductive.

For these reasons, it is desirable to provide devices and methods for providing IV set components and interfaces with light protective properties and that are translucent to provide visibility to the fluid path or level within the component or interface, thus providing visibility to the fluid path or level within the component or interface while protecting the fluid from light exposure.

### SUMMARY

In one or more embodiments, a light protective infusion set component, comprises an infusion set component, wherein a portion of the infusion set component is translucent, providing visibility within the infusion set component; and a layer of flexible light protective material having a filter for a predetermined range of light wavelengths, wherein the layer of flexible light protective material is disposed around an exterior surface of the translucent portion of the infusion set component.

In one or more embodiments, a light protective infusion set layer, comprises a layer of flexible translucent material; a filter for a predetermined range of light wavelengths; and an adhesive component, wherein the layer of flexible translucent material is configured to be disposed around an exterior surface of a translucent portion of an infusion set component.

In one or more embodiments, a method of forming a light protective infusion set component comprises removing a portion of light protective infusion set layer material from a light protective infusion set layer supply, wherein the light protective infusion set layer material is configured to filter a predetermined range of light wavelengths; preparing the portion of light protective infusion set layer material for attachment to an infusion set component having a translucent portion, wherein the light protective infusion set layer material comprises a self-sticking property; and forming the portion of light protective infusion set layer material over an outer surface of the translucent portion of the infusion set component.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIGS. 2A-2E are front views of a typical assembled infusion set and typical infusion set components.

FIG. 3 is a front view of an example foil wrapped infusion set.

FIG. 4 depicts a top view of an example opaque infusion set.

FIG. 5 depicts a top view of an infusion set layer, according to aspects of the disclosure.

FIG. 6 depicts a perspective view of an infusion set layer, according to aspects of the disclosure.

FIG. 7 depicts a perspective view of an infusion set layer, according to aspects of the disclosure.

FIG. 8 depicts a schematic view of an infusion set layer, according to aspects of the disclosure.

FIG. 9 depicts a front view of an infusion set layer, according to aspects of the disclosure.

FIGS. 10A and 10B depict top view of the infusion set layer of FIG. 9 in open and closed positions, according to aspects of the disclosure.

FIG. 11 depicts a perspective view of an infusion set layer dispenser, according to aspects of the disclosure.

FIG. 12 illustrates a method of adding a light protective infusion set layer to an infusion set component, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

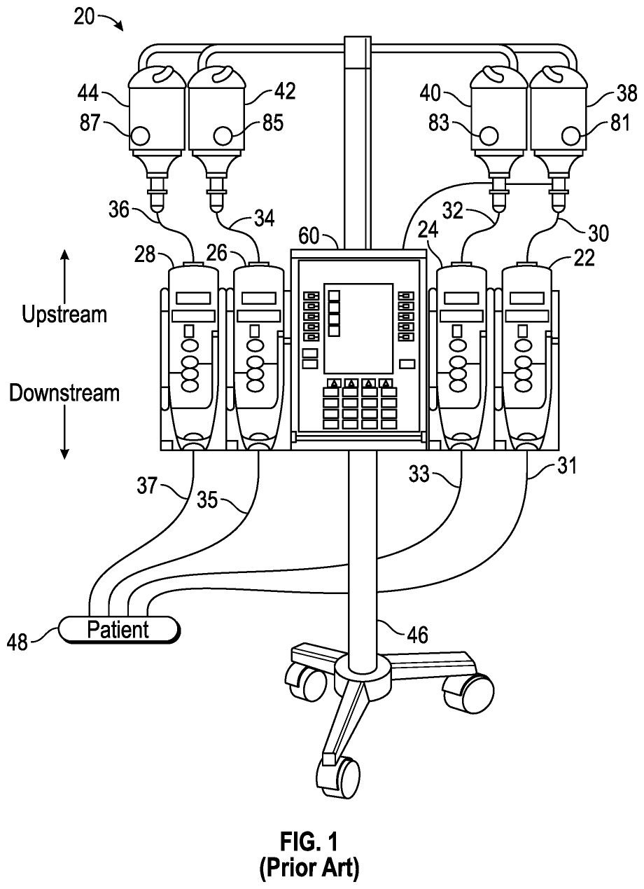

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter. For example, the pump control unit 60 may receive information from the electronic data tag 83 identifying a fluid to be infused by the infusion pump 24. The pump control unit 60 may include or receive configuration to ensure safe administration of the fluid. If the fluid is identified as a light sensitive fluid, the configuration may cause an adjustment to one or more elements of the patient care system 20. The adjustment may pause or prevent administration of the fluid until certain safety precautions are taken and/or detected. One such safety precaution is protecting at least a portion of the infusion line with a light shielding layer. In some implementations, the patient care system 20 may infer protection of the line through the scanning of an identifier for the light protecting device or material. For example, a clinician may scan a barcode on protective light film before applying to the infusion line. The scanned information may be accessible by the patient care system 20 to infer the protection of the infusion line. The scanned information may indicate a spectrum of protection provided by the scanned element. In some implementations, the patient care system 20 may additionally or alternatively confirm that the spectrum of protection provided will prevent wavelengths of harm for the fluid to be infused. That is, an additional check may be performed to ensure that the line is not only protected by some light protective covering but also protected with an appropriate light protective covering for the fluid. Additional or alternative confirmations may be performed such as image detection of the infusion line to detect through, for example, image or color analysis, that the line is protected, or weight or load detection via a sensor measuring the infusion administration line to determine whether a detected weight or load corresponds to an expected weight or load of the infusion set with an applied light protection layer.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

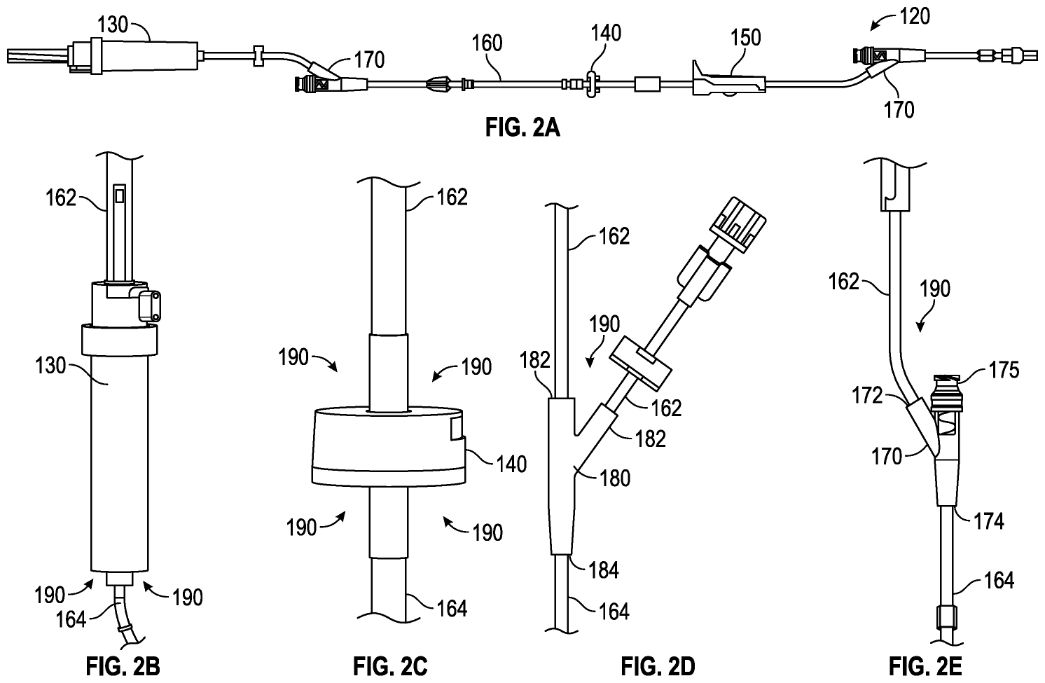

Typically, medical fluid administration sets have more parts than are shown in FIG. 1, such as those shown in FIGS. 2A-2D and 4. Infusion sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIGS. 2A-2E, an infusion set 120 may include a drip chamber 130, a check valve 140 and a roller clamp 150 connected together by tubing 160. Infusion set 120 may also include a Y-site 170 having a Y-shaped junction with an inlet port 172 connected to an inlet tube 162, a needleless connector 175 and an outlet port 174 connected to an outlet tube 164. The needleless connector 175 provides a port for the introduction of fluid into and/or removal of air bubbles from the infusion set 120. For example, a needleless syringe (not shown) may be connected to the needleless connector 175 in order to add medication to a saline fluid that is gravity fed from a saline bag (not shown) into the drip chamber 130. The infusion set 120 may include additional infusion components and may be formed of any combination of components and the tubing 160.

As shown in more detail in FIG. 2B, the drip chamber 130 is connected to an inlet tube 162 and an outlet tube 164. Similarly, in FIG. 2C the check valve 140 is connected to an inlet tube 162 and an outlet tube 164. FIG. 2D shows a Y-junction 180 having two inlet ports 182 and one outlet port 184, where each inlet port 182 is connected to an inlet tube 162 and the outlet port 184 is connected to an outlet tube 164. The Y-site 170 shown in FIG. 2E has an inlet port 172 connected to an inlet tube 162, an outlet port 174 connected to an outlet tube 164 and a needleless connector 175.

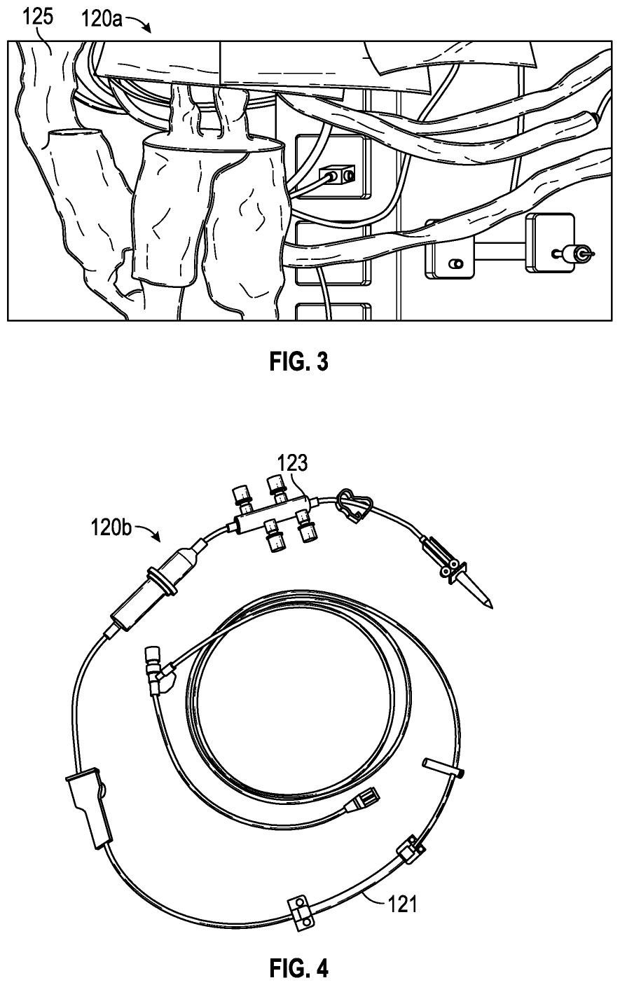

As shown in FIG. 3, a typical solution for providing light protection to an infusion set 120a is to wrap the components in aluminum foil 125. Here, every component and tube of the infusion set 120a is fully wrapped with the foil 125, rendering the entire infusion set 120a completely opaque and allowing no visibility as to the internal fluid flow or level at all. Further, the addition of the metal of foil 125 provides an increased risk of electrostatic discharge or undesired electrical conduction to/from the pump or other sensitive equipment used in various healthcare settings, such as operating rooms, intensive care units and hospital rooms.

As shown in FIG. 4, another typical solution is to provide most or all of an infusion set 120b with opaque materials. However, in the case of the entire infusion set 120b being opaque, this again allows no visibility as to the internal fluid flow or level at all. In some cases, certain components of infusion set 120b (e.g., extension 121, fluid manifold 123) may be translucent in order to provide some visibility as to the fluid flow path or level within that component. However, this solution again allows for the fluid within these translucent components to be exposed to undesired light ranges, thus degrading or transforming the fluid.

In aspects of the disclosure, a translucent light shielding layer is provided for protection of infusion set components and tubing from undesired light exposure. In aspects of the disclosure, the light shielding layer may be provided in any desired form, such as a sheet of material, a roll of adhesive material, a roll of clinging material, and the like. In aspects of the disclosure, the translucent light shielding layer has desired medical properties, such as sterility, ability to be sprayed with fluids and/or wiped down with cleaning agents, antimicrobial properties, antifungal properties, and the like. The light shielding layer may filter out or block any desired light range. For example, one medical standard requires that fluid components may not transmit or allow to pass through more than 35 percent of light in the 250-440 nanometer range (e.g., ultraviolet (UV) light). In another example, some drugs are sensitive to light ranges above the UV range (e.g., visible light spectrum such as 520 nanometer range), where the light shielding layer is configured to filter out or block those higher wavelength light levels.

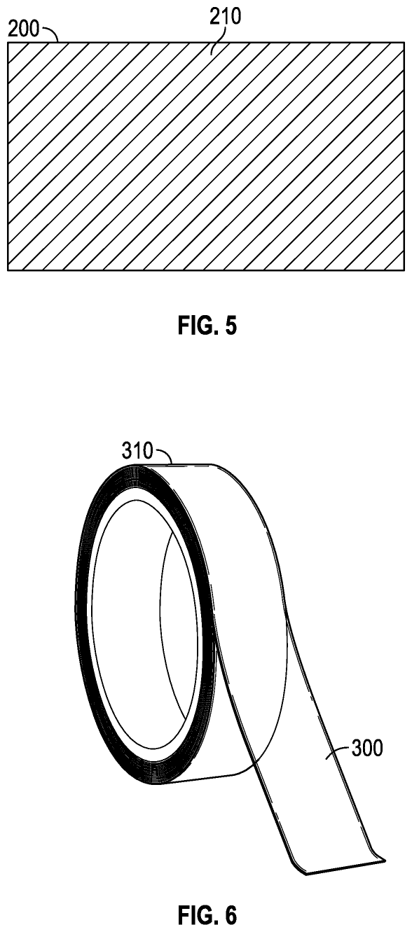

Turning now to FIG. 5, an infusion set layer 200 is shown according to aspects of the disclosure. Infusion set layer 200 may be a sheet 210 of translucent light shielding material that may be cut or shaped to fit around desired infusion set components (e.g., fluid supply 38, drip chamber 130, tubing 160, inlet tube 162, outlet tube 164, Y-junction 180). In some implementations, the sheet 210 may be perforated to allow separation into uniform portions corresponding to one or more elements of an infusion administration set. Infusion set layer 200 may be colored for easy identification of which components in an infusion set (e.g., infusion set 120) are covered or wrapped by some or all of infusion set layer 200. Whether colored or clear, infusion set layer 200 is translucent so that fluid paths or levels of the associated infusion set component are visible. For example, when drip chamber 130 includes an infusion set layer 200 covering the external surface of the drip chamber 130, the drip rate of fluid into the drip chamber 130 and the fluid level contained in the drip chamber 130 are visible, thus allowing efficient and effective use of the drip chamber 130 while providing protection against a particular range of light exposure to the fluid within the drip chamber 130.

Similarly in FIG. 6, an infusion set layer 300 is shown according to aspects of the disclosure. Infusion set layer 300 may be a tape roll 310 of translucent light shielding material that may be wrapped around desired infusion set components (e.g., fluid supply 38, drip chamber 130, tubing 160, inlet tube 162, outlet tube 164, Y-junction 180). For example, the tape roll 310 may have 1.5 yards to 10 yards of infusion set layer 300 tape having a width of 0.5, 1, 2 or 3 inches. Infusion set layer 300 may be colored for easy identification of which components in an infusion set (e.g., infusion set 120) are covered or wrapped by infusion set layer 300. Whether colored or clear, infusion set layer 300 is translucent so that fluid paths or levels of the associated infusion set component are visible. For example, when tubing 160 includes an infusion set layer 300 covering the external surface of the tubing 160, the flow rate or level of fluid in the tubing 160 are visible, thus allowing efficient and effective use of the tubing 160 while providing protection against a particular range of light exposure to the fluid within the tubing 160. In some aspects of the disclosure, infusion set layer 300 may be wrapped around the desired infusion set component in multiple layers to increase light shielding protection.

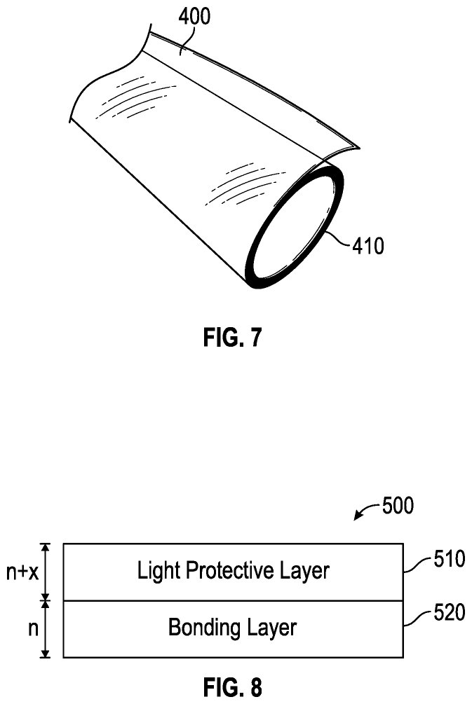

Turning now to FIG. 7, an infusion set layer 400 is shown according to aspects of the disclosure. Infusion set layer 400 may be a wrap roll 410 of translucent light shielding material that may be cut, torn and/or shaped to fit around desired infusion set components (e.g., fluid supply 38, drip chamber 130, tubing 160, inlet tube 162, outlet tube 164, Y-junction 180). Infusion set layer 400 may be colored for easy identification of which components in an infusion set (e.g., infusion set 120) are covered or wrapped by some or all of infusion set layer 400. Whether colored or clear, infusion set layer 400 is translucent so that fluid paths or levels of the associated infusion set component are visible. For example, when a Y-junction assembly (e.g., Y-junction 180, inlet tube 162, outlet tube 164) includes an infusion set layer 400 covering the external surface of the Y-junction assembly, the fluid flows into and out of the Y-junction 180 through the inlet tube 162 and outlet tube 164 are visible, thus allowing efficient and effective use of the Y-junction assembly while providing protection against a particular range of light exposure to the fluid within the Y-junction assembly.

An infusion set layer 500 may have multiple layers as shown in FIG. 8. For example, the infusion set layer 500 may have a light protective layer 510 and a bonding layer 520. The light protective layer 510 may have any suitable thickness x for obtaining the desired level of light shielding. The bonding layer 520 may have any suitable thickness n for providing the desired level of adherence to an infusion component surface. Accordingly, the thickness of the infusion set layer 500 may be the combination of the light protective layer 510 thickness x and the bonding layer 520 thickness n (e.g., n+x).

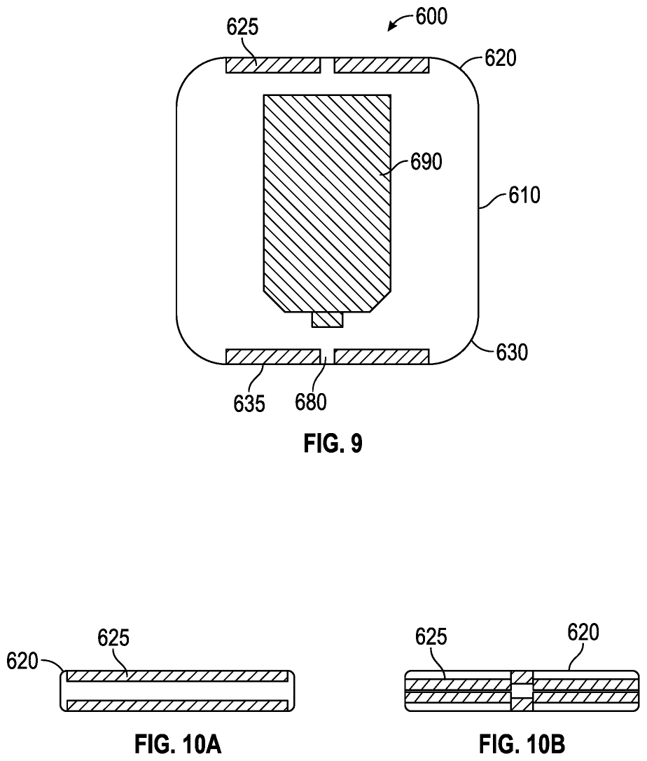

Turning to FIG. 9, an infusion set layer 600 may be provided as a preformed sealable bag 610 suitable for placing around an infusion set component (e.g., infusion container 690). The bag 610 may have an openable top 620, an openable bottom 630 or both, providing flexibility for placing the infusion set component within the bag 610. The top 620 may have a sealing section 625 that may be any suitable sealing device, such as an adhesive bonding, a plastic zipper, a loop and hook combination (e.g., Velcro®), a pressure sensitive adhesive, and the like. The bottom 630 may have a sealing section 635 having any suitable sealing device similarly or differently than the sealing section 625. If the sealing section 625 and sealing section 635 are different sealing devices, a fiduciary marking may be included on bag 610 to provide a visual indication of which side of the bag 610 is the top 620. The bottom 630 may be attached to an infusion line 680, attached to a bottom portion of the infusion container 690, or sealed against itself with no infusion set component protruding out of the bottom 630. Similarly, the top 620 may be attached to a top portion of the infusion container 690, sealed against itself with no infusion set component protruding out of the top 620, or attached to an infusion line coming out of the top 620 (not shown).

FIGS. 10A and 10B show top views of the bag 610 in an open position (e.g., sealing section 625 not sealed) and in a sealed position (e.g., sealing section 625 sealed) respectively. For example, in the open position, two halves of the sealing section 625 may be separated, thus allowing for the top 620 to be opened, whereas in the closed position the two halves of the sealing section 625 may be joined, thus sealing the top 620.

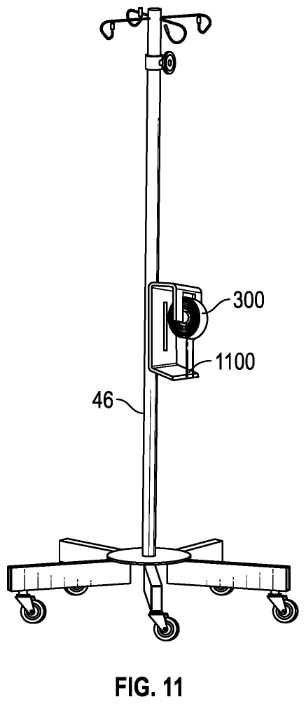

FIG. 11 shows a dispenser 1100 attached to an IV pole 46. The dispenser 1100 is configured to hold an infusion set layer 300 in the form of a tape roll (e.g., tape roll 310). In aspects of the disclosure, any suitable form of dispensing may be provided for infusion set layers 200, 300, 400, 500, 600 may be provided. For example, butcher paper, a dispenser box with a foil edge, a mounted roll, adhesive, a bag dispenser, a clingy material roll dispenser, and the like. The infusion set layer 200, 300, 400, 500, 600 may be pulled out and severed by a cutting edge of a dispenser, cut with scissors or a knife, easily torn and the like.

In aspects of the disclosure, the infusion set layer 200, 300, 400, 500, 600 may be configured or designed to be easily removable, such as if it gets stuck on a pump surface when being applied, for example. The infusion set layer 200, 300, 400, 500, 600 may be flexible, not dissolvable if it comes into contact with infusate or cleaning supplies, and compliant (e.g., adaptable) to the contours of the infusion set component or tubing (e.g., allow for ambulatory movement). In aspects of the disclosure, the infusion set layer 200, 300, 400, 500, 600 may include writing, such as to identify set loading, dimensions, type of protection provided, and the like.

In some aspects of the disclosure, an infusion set layer may be provided as a pre-formed covering for a specific infusion set component. For example, the material of infusion set layer 200 may be formed into a sleeve or a bag that is sized and shaped to fit over a predetermined infusion set component (e.g., fluid source 38, drip chamber 130). In aspects of the disclosure, a predetermined amount of an infusion set layer material (e.g., infusion set layer 200, 300, 400, 500, 600) may be packaged with an infusion set (e.g., infusion set 120), such as a determined amount of infusion set layer material needed to protect the required elements of the infusion set, for example.

In aspects of the disclosure, an infusion set layer may have an adhesive component. For example, the infusion set layer 200 may have an adhesive coating on one side with a peel away covering over the adhesive coating. Here, the covering may be peeled away and the side with the adhesive coating placed in contact with the desired surfaces of the infusion set component, thus keeping the infusion set layer 200 in place. In another example, infusion set layer 300 may have an adhesive coating on an inner layer of the tape. Here, a section of the tape may be pulled out from the tape roll 310, and the desired length of infusion set layer 300 may be wrapped around an infusion set component.

In aspects of the disclosure, an infusion set layer may be a wrap with a clingy property. For example, the infusion set layer 400 may be formed of a lightweight clingy wrap material that sticks to itself. Here, the a section of the wrap may be pulled out from the wrap roll 410, and the desired section of infusion set layer 400 may be wrapped around an infusion set component, where portions of the infusion set layer 400 that come into contact with each other stick together.

In aspects of the disclosure, the infusion set layer 200, 300, 400, 500, 600 may be used to cover portions of a syringe. For example, an infusion set layer 200, 300, 400, 500 may be wrapped around the syringe. As another example, an infusion set layer 600 may be a bag 610 in which the entire syringe or syringe module is placed.

In aspects of the disclosure, infusion set layers 200, 300, 400, 500, 600 may have different colors to help differentiate infusion set lines, such as complex infusion setups in an operating room or intensive care unit, for example. Different colors may also provide different levels of light shielding protection. For example, yellow coloring may provide low light shielding protection, while orange, red or brown may provide higher levels of light shielding protection. In aspects of the disclosure, UV absorbers may be added to any infusion set layer 200, 300, 400, 500, 600 and any color for increased light shielding protection. The coloring and levels of light shielding protection may be designed for preventing certain wavelengths of light from reaching the infusion fluid within the infusion set. For example, the ultraviolet spectrum (typically 250 nm to 450 nm) may be filtered by one or more of the layers described.

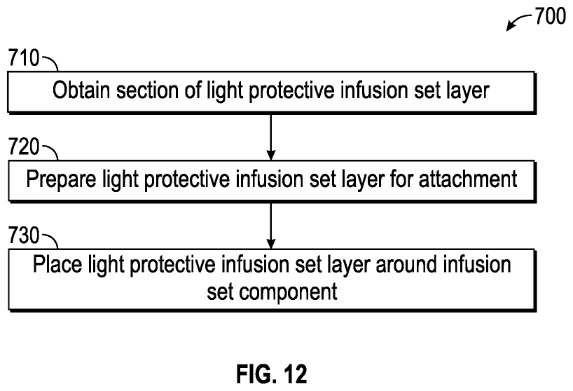

According to some aspects of the disclosure, a method 700 of forming a light protective infusion set component is shown in FIG. 12. In step 710, a section of a light protective infusion set layer (e.g., infusion set layer 200, 300, 400, 500, 600) is obtained (e.g., cut from sheet 210, severed from tape roll 310, severed from wrap roll 410, removed from a bag 610 container). Here, the light protective infusion set layer is obtained in a desired size or length for an identified infusion set component (e.g., fluid supply 38, drip chamber 130, tubing 160, inlet tube 162, outlet tube 164, Y-junction 180).

The light protective infusion set layer is prepared for attachment to the infusion set component in step 720. For example, a cover layer may be removed from an adhesive coating on the light protective infusion set layer. As another example, an adhesive side of the light protective infusion set layer may be disengaged from a storage surface (e.g., a non-adhesive side of the light protective infusion set layer on a roll). As yet another example, the light protective infusion set layer may be disengaged from a storage surface (e.g., the light protective infusion set layer may be unstuck from itself on a roll). As another example, a bag 610 may be opened to receive the infusion set component.

In step 730, the light protective infusion set layer is placed around the outer surface(s) of the infusion set component. For example, the light protective infusion set layer may be formed around the infusion set component (e.g., infusion set layer 200 formed around component) or the light protective infusion set layer may be wrapped around the infusion set component (e.g., infusion set layer 300 tape or infusion set layer 400 wrap wound around the infusion set component). Here, an adhesive coating or clingy property of the light protective infusion set layer causes the light protective infusion set layer to adhere, stick or cling to the outer surface(s) of the infusion set component and/or other portions of the light protective infusion set layer. The adhesive coating may include or be formed with polyvinyl chloride, low density polyethylene, linear low density polyethylene, or similar plastic or thermoplastic material. In another example, the light protective infusion set layer 600 may be a bag 610 or pouch that is pulled or slid over the infusion set component, where the light protective infusion set layer 600 may not have an adhesive layer or clingy property over the entire bag 610, but just at the sealable sections 625, 635 at the top 620 and/or bottom 630.

In one or more embodiments, a light protective infusion set component comprises an infusion set component, wherein a portion of the infusion set component is translucent, providing visibility within the infusion set component; and a layer of flexible light protective material having a filter for a predetermined range of light wavelengths, wherein the layer of flexible light protective material is disposed around an exterior surface of the translucent portion of the infusion set component.

In aspects of the disclosure, the filter is configured to allow no more than 35 percent of ultraviolet light to pass through the layer of flexible light protective material. In aspects of the disclosure, the filter is configured to block ultraviolet light from passing through the layer of flexible light protective material. In aspects of the disclosure, an adhesive layer is disposed on one side of the layer of flexible light protective material. In aspects of the disclosure, the layer of flexible light protective material comprises a self-sticking property. In aspects of the disclosure, the layer of flexible light protective material comprises a colored pigment.

In aspects of the disclosure, the layer of flexible light protective material is translucent. In aspects of the disclosure, the layer of flexible light protective material is formed of a material rated for infusion procedures. In aspects of the disclosure, the infusion set component is intravenous tubing. In aspects of the disclosure, the infusion set component is a fluid source container. In aspects of the disclosure, the infusion set component is a drip chamber. In aspects of the disclosure, the infusion set component is a Y-junction.

In one or more embodiments, a light protective infusion set layer, comprises a layer of flexible translucent material; a filter for a predetermined range of light wavelengths; and an adhesive component, wherein the layer of flexible translucent material is configured to be disposed around an exterior surface of a translucent portion of an infusion set component.

In aspects of the disclosure, the adhesive component is one of an adhesive layer disposed on one side of the layer of flexible translucent material and a self-sticking property of the layer of flexible translucent material. In aspects of the disclosure, the filter is configured to allow no more than 35 percent of ultraviolet light to pass through. In aspects of the disclosure, the layer of flexible translucent material is colored. In aspects of the disclosure, the layer, the filter, and the adhesive component are arranged to form a sheet material. In aspects of the disclosure, the adhesive component is disposed on a first side of the light protective infusion set layer, and wherein the layer, the filter, and the adhesive component are arranged to form a single sided tape material. In aspects of the disclosure, the adhesive component is disposed on a first side of the light protective infusion set layer, and wherein the adhesive component comprises at least one of polyvinyl chloride or low density polyethylene, and wherein the layer, the filter, and the adhesive component are arranged to form cling wrap material.

In one or more embodiments, a method of forming a light protective infusion set component comprises removing a portion of light protective infusion set layer material from a light protective infusion set layer supply, wherein the light protective infusion set layer material is configured to filter a predetermined range of light wavelengths; preparing the portion of light protective infusion set layer material for attachment to an infusion set component having a translucent portion, wherein the light protective infusion set layer material comprises a self-sticking property; and forming the portion of light protective infusion set layer material over an outer surface of the translucent portion of the infusion set component.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

1. A light protective infusion set component, comprising:
an infusion set component, wherein a portion of the infusion set component is translucent, providing visibility within the infusion set component; and
a layer of flexible light protective material having a filter for a predetermined range of light wavelengths,
wherein the layer of flexible light protective material is disposed around an exterior surface of the translucent portion of the infusion set component.

2. The light protective infusion set component of claim 1, wherein the filter is configured to allow no more than 35 percent of ultraviolet light to pass through the layer of flexible light protective material.

3. The light protective infusion set component of claim 1, wherein the filter is configured to block ultraviolet light from passing through the layer of flexible light protective material.

4. The light protective infusion set component of claim 1, further comprising:
an adhesive layer disposed on one side of the layer of flexible light protective material.

5. The light protective infusion set component of claim 1, wherein the layer of flexible light protective material comprises a self-sticking property.

6. The light protective infusion set component of claim 1, wherein the layer of flexible light protective material comprises a colored pigment.

7. The light protective infusion set component of claim 1, wherein the layer of flexible light protective material is translucent.

8. The light protective infusion set component of claim 1, wherein the layer of flexible light protective material is formed of a material rated for infusion procedures.

9. The light protective infusion set component of claim 1, wherein the infusion set component is intravenous tubing.

10. The light protective infusion set component of claim 1, wherein the infusion set component is a fluid source container.

11. The light protective infusion set component of claim 1, wherein the infusion set component is a drip chamber.

12. The light protective infusion set component of claim 1, wherein the infusion set component is a Y-junction.

13. A light protective infusion set layer, comprising:
a layer of flexible translucent material;
a filter for a predetermined range of light wavelengths; and
an adhesive component,
wherein the layer of flexible translucent material is configured to be disposed around an exterior surface of a translucent portion of an infusion set component.

14. The light protective infusion set layer of claim 13, wherein the adhesive component is one of an adhesive layer disposed on one side of the layer of flexible translucent material and a self-sticking property of the layer of flexible translucent material.

15. The light protective infusion set layer of claim 13, wherein the filter is configured to allow no more than 35 percent of ultraviolet light to pass through.

16. The light protective infusion set layer of claim 13, wherein the layer of flexible translucent material is colored.

17. The light protective infusion set layer of claim 13, wherein the layer, the filter, and the adhesive component are arranged to form a sheet material.

18. The light protective infusion set layer of claim 13, wherein the adhesive component is disposed on a first side of the light protective infusion set layer, and wherein the layer, the filter, and the adhesive component are arranged to form a single sided tape material.

19. The light protective infusion set layer of claim 13, wherein the adhesive component is disposed on a first side of the light protective infusion set layer, and wherein the adhesive component comprises at least one of polyvinyl chloride or low density polyethylene, and wherein the layer, the filter, and the adhesive component are arranged to form cling wrap material.

20. A method of forming a light protective infusion set component, the method comprising:
removing a portion of light protective infusion set layer material from a light protective infusion set layer supply, wherein the light protective infusion set layer material is configured to filter a predetermined range of light wavelengths;
preparing the portion of light protective infusion set layer material for attachment to an infusion set component having a translucent portion, wherein the light protective infusion set layer material comprises a self-sticking property; and
forming the portion of light protective infusion set layer material over an outer surface of the translucent portion of the infusion set component.
